(19) United States
(12) Patent Application Publication
(10) Pub. No.: US 2011/0117660 A1
(43) Pub. Date: May 19, 2011

(54) PHYSICAL CHARACTERIZATION OF OLIGONUCLEOTIDE CONJUGATES

(76) Inventor: Douglas G. BROOKS, Durham, NC (US)

(21) Appl. No.: 12/619,602

(22) Filed: Nov. 16, 2009

(51) Int. Cl. G01N 33/48; C07H 1/00

(52) U.S. Cl. 436/94; 536/25.4

(57) ABSTRACT

The present invention relates generally to methods useful in characterizing oligonucleotide conjugates, comprising an oligonucleotide and a modifying high molecular weight compound, by providing means for separating the high molecular weight compound from the oligonucleotide while maintaining the oligonucleotide's structural integrity. The ability to break the oligonucleotide construct down into its constituent parts while maintaining the structural integrity of the oligonucleotide allows for more efficient and effective downstream analysis including, but not limited to, sequencing of the oligonucleotide, characterization of degradation products, and identification of areas of decreased stability in the oligonucleotide conjugate.

### 1. FIELD OF INVENTION

The present invention relates generally to methods useful in characterizing oligonucleotide conjugates, comprising an oligonucleotide and a modifying high molecular weight compound, by providing means for separating the modifying high molecular weight compound from the oligonucleotide while maintaining the oligonucleotide's structural integrity.

### 2. BACKGROUND OF THE INVENTION

Oligonucleotide-based therapeutics continue to show increasing promise as a viable class of therapeutics for use in treating a number of human disorders. Despite many favorable characteristics, oligonucleotide therapeutics can sometimes be limited by less than desirable cellular uptake profiles, limited biological stability, or a short half-life resulting in less than optimal pharmacokinetics. Hence, it may be challenging to maintain a given oligonucleotide therapeutic at a desired concentration in the blood sufficient to exert a desired physiological effect.

To date, the most widely used approaches to obtain improved stability, distribution and pharmacokinetics of therapeutic oligonucleotides comprise: 1) modifying the oligonucleotide compound to increase circulating half-life and; 2) encapsulating the therapeutic compound in, for example, polymer microspheres, liposomes, or polymer micelles. In connection with the first approach, conjugation of therapeutic oligonucleotides with hydrophobic moieties is one way to improve the in vivo stability and half-life of a given therapeutic oligonucleotide. Biocompatible polymers that can be used for this purpose include, but are not limited to, polyalkylene glycols (PAGs), polylactide/glycolide polymers (PLGA), and polyaminoamine (PAMAM). PAGs are especially well suited to conjugation with therapeutic compounds—including oligonucleotides—and the use and safety of PAGS conjugated to therapeutic compounds has been well documented in vivo (Zalipsky Bioconjugate Chem, 6:150-165, 1995 and Adv Drug Delivery Rev, 16:157-182, 1995; T. G. McCauley et al. Pharm. Res. 23:303, 2006). In particular, therapeutic oligonucleotides are often conjugated with polyethylene glycol (PEG), such as methoxy-PEG (mPEG), to achieve extended in vivo circulation as compared to unconjugated biopolymers.

In the process of making oligonucleotides modified with hydrophobic moieties, it is often desirable to be able to characterize both the intermediate and the final product. For example, in manufacturing a therapeutic product, it is essential to be able to detect the presence of impurities generated or introduced in the process of making the final product. Further, it is desirable to be able to detect areas of instability as well as measure other physical characteristics in order to improve the stability and/or specificity of the oligonucleotide. The presence of the high molecular weight compound, however, may limit the observable physical characteristics of the oligonucleotide conjugate itself. For example, the presence of a high molecular weight compound interferes with the ability to sequence the oligonucleotide, reduces chromatographic separation of the construct due to the polydispersity of the high molecular weight compound, and makes determination of the construct's molecular weight difficult as the weight percent of the oligonucleotide can vary between lots of some hydrophobic moieties. Owing in part to the above limitations, the presence of a high molecular weight compound also makes it difficult to resolve impurities using standard analytical methods such as high performance liquid chromatography, mass spectroscopy, or nuclear magnetic resonance.

One way to address the above limitations is to remove the high molecular weight compound from the final product in order to allow more effective and efficient characterization of the oligonucleotide component. For example, the oligonucleotide can be sequenced to insure a proper nucleotide sequence. The oligonucleotide can also be analyzed using mass spectroscopy and nuclear magnetic resonance to determine the presence of non-specific oligonucleotide synthesis products, degradation products, or other potential impurities. Due to the tendency of glycosidic and phosphodiester bonds within an oligonucleotide to undergo cleavage when exposed to acidic and/or basic conditions, reaction conditions optimal for cleavage of the high molecular weight compound, however, can lead to unwanted cleavage or modification of the oligonucleotide.

As such, standard methods for cleaving hydrophobic moieties from oligonucleotides may disrupt the oligonucleotides structural integrity, and are therefore not particularly useful in analyzing the quality of the final product. Therefore, what is needed in the art are methods that allow for cleavage of hydrophobic moieties from oligonucleotides while maintaining the oligonucleotide's structural integrity.

### 3. SUMMARY OF INVENTION

The present invention relates generally to methods for separating oligonucleotide conjugates into their component parts. More specifically, the present invention provides a method for removing a high molecular weight compound from an oligonucleotide in a controlled manner that maintains the oligonucleotide's structural integrity. The ability to break the oligonucleotide conjugate down into its constituent parts while maintaining the structural integrity of the oligonucleotide allows for more efficient and effective downstream analysis including, but not limited to, sequencing of the oligonucleotide, characterization of degradation products, and identification of areas of decreased stability in the oligonucleotide conjugate.

Oligonucleotides that may be analyzed or processed according to the present invention include, but are not limited to, siRNAs, antisense oligonucleotides, aptamers and other structural oligonucleotides such as ribozymes. In one exemplary embodiment, the oligonucleotide is an aptamer. Oligonucleotides may also comprise unmodified, modified, or a combination of unmodified and modified nucleotides. In one exemplary embodiment the oligonucleotides comprise at least one modified nucleotide. In another exemplary embodiment, the oligonucleotides comprises 2′-fluoro and 2′-O-methyl modified nucleotides.

High molecular weight compounds that may be removed from an oligonucleotide construct of the present invention include, but are not limited to, polyalkylene glycols, such as polyethylene glycol (PEG); polylactide/glycolde polymers (PLGA); polyaminoamines (PAMAM); polysaccharides, such as dextran; and polyoxazolines. In one exemplary embodiment, the high molecular weight compound comprises a linear PEG. In another exemplary embodiment, the high molecular weight compound comprises a branched PEG. In yet another exemplary embodiment, the high molecular weight compound comprises a polyoxazolines.

Oligonucleotide conjugates for use with the present invention may comprise a high molecular weight compound connected to an oligonucleotide via a linker. Suitable linkers may comprise carbamate or ester functionalities. In one exemplary embodiment the linker comprises a carbamate functional group. In one exemplary embodiment, the carbamate group links the high molecular weight compound to a core, such as glycine or glycerol. In another exemplary embodiment, the carbamate group is located intermediate to the high molecular weight compound and the oligonucleotide.

In one exemplary embodiment, the methods of the present invention comprise mixing a composition comprising an oligonucleotide conjugate with a cleavage reagent to form a reaction composition and incubating the reaction composition for a time and at a temperature sufficient to bring about cleavage of the oligonucleotide conjugate into separable cleavage products. In one exemplary embodiment, the cleavage products comprise a high molecular weight compound cleavage product and an oligonucleotide cleavage product. In another exemplary embodiment, the oligonucleotide cleavage product comprises one or more detectable oligonucleotide cleavage species. The one or more detectable oligonucleotide cleavage species may comprise one or more residual functional groups at the site of conjugation. In one exemplary embodiment, the residual functional group is a carboxy group. The residual functional groups provide the oligonucleotide cleavage product with a distinctive mass that can be utilized to distinguish the cleavage product from non-conjugate oligonucleotides or other impurities using standard analytical techniques. After incubation is complete, the oligonucleotide may be separated from the polymer using liquid chromatography, gel electrophoresis, or any other suitable separation method known in the art. The oligonucleotide may then subjected to further analysis, including but not limited to, sequencing, and structural analysis using mass spectroscopy and/or nuclear magnetic resonance.

The cleavage reagent may comprise a hydroxide. The concentration of the hydroxide may range from about 0.15 to 1 M. In one exemplary embodiment the concentration ranges from about 0.16 to about 0.85 M. In another exemplary embodiment, the concentration ranges from about 0.25 to about 0.75 M. In one exemplary embodiment the hydroxide is an alkali metal or an alkaline metal hydroxide. In another exemplary embodiment, the hydroxide is a sodium hydroxide.

The reaction composition may be maintained at a temperature ranging from about 20 to about 60° C. In one exemplary embodiment the temperature ranges from about 20 to about 40° C. In another exemplary embodiment the temperature ranges from about 25 to about 40° C. In yet another exemplary embodiment, the temperature ranges from about 30 to about 40° C.

In one exemplary embodiment, the present invention is directed to a method of analyzing a sample comprising an oligonucleotide conjugate. In one exemplary embodiment the sample is from a manufactured batch of oligonucleotide conjugate. In another exemplary embodiment, the sample is a body fluid sample from a subject previously administered an oligonucleotide conjugate. The method of analyzing a sample containing an oligonucleotide conjugate comprises: mixing the sample with a cleavage reagent to form a reaction composition; incubating the reaction composition for a time and at a temperature sufficient to generate a high molecular weight compound cleavage product and an oligonucleotide cleavage product, wherein the oligonucleotide cleavage product comprises one or more oligonucleotide cleavage product species; separation of the oligonucleotide cleavage product, or products from the high molecular weight compound cleavage product; and analysis of the oligonucleotide cleavage product, or products.

The methods of the present invention may optionally include a preliminary purification step to removed non-conjugated oligonucleotides from the sample. In the case where the sample is a biological sample, the sample may also be optionally purified to remove potential interfering substances. Standard purification techniques known in the art, such as liquid chromatography may be used.

In one exemplary embodiment analysis of the oligonucleotide cleavage product comprises sequencing of the oligonucleotide cleavage product.

In another exemplary embodiment analysis of the oligonucleotide cleavage product comprises detection of degradation products using mass spectroscopy or NMR. In one exemplary embodiment, detection of degradation products comprises detecting the presence or absence of one or more residual functional groups present on one or more of the oligonucleotide cleavage product species, wherein detection of the presence of the residual functional group indicates the species was conjugated to a high molecular weight compound, and wherein the absence of the residual functional group indicates the species was not conjugated to a high molecular weight compound. In one exemplary embodiment, the residual functional group is a carboxy group. In another exemplary embodiment, failure to detect a residual functional group on an oligonucleotide cleavage species indicates the presence of an impurity.

In one exemplary embodiment of the present invention, the method comprises: mixing a purified aptamer conjugate, wherein the aptamer conjugate comprises an aptamer conjugated to a high molecular weight compound via a linker, with a cleavage reagent to form a reaction composition; incubating the reaction composition for a time and at a temperature sufficient to generate a high molecular weight compound cleavage product and an aptamer cleavage product, wherein the aptamer cleavage product comprises one or more aptamer cleavage product species; separation of the aptamer cleavage product, or products from the high molecular weight compound cleavage product; and analysis of the aptamer cleavage products.

Analysis of the aptamer cleavage product may comprise sequencing the aptamer cleavage products and/or detection of degradation products using mass spectroscopy. In one exemplary embodiment, the high molecular weight compound comprises a branched PEG, wherein each PEG arm is connected to a central core via a linker comprising a carbamate group. In another exemplary embodiment, the carbamate group is intermediate to the high molecular weight compound and the oligonucleotide.

In one embodiment, the aptamer comprises modified nucleotides and analysis comprises detection of degradation products via mass spectroscopy. Observed aptamer cleavage product species include terminally modified fragments containing a carboxy group at the 5′ terminus, the 3′ terminus, or both, and terminally unmodified cleavage products. The size of the terminally unmodified cleavage products can be determined, for example, the number and location of unmodified nucleotides in the oligonucleotide. The detection of additional aptamer cleavage product species beyond the expected aptamer cleavage product species indicates degradation of the aptamer conjugate. The degradation products may be further characterized, for example, by sequencing in order to determine where in the oligonucleotide the lesions are occurring.

In one embodiment, the structural integrity of an aptamer conjugate is confirmed by mixing the aptamer conjugate with a cleavage reagent to form a reaction mixture; incubating the reaction mixture at suitable conditions to generate one or more high molecular weight cleavage products and an aptamer cleavage products; separating the aptamer cleavage product form the high molecular weight product; and analyzing the cleavage product for the presence of one or more degradation products in the aptamer cleavage product, wherein the detection of degradation products indicates degradation of the aptamer.

### 4. BRIEF DESCRIPTION OF THE DRAWINGS

FIG. 1 is diagram depicting exemplary cleavage products of an exemplary oligonucleotide conjugate according to the method of the present invention.

FIG. 2 depicts the use of selective excitation to suppress the water signal in order to address presence of a high molecular weight compound in the NMR spectra of an oligonucleotide conjugate.

FIG. 3 is a mass spectrum showing the distribution of expected cleavage products according to one exemplary embodiment of the invention.

FIG. 4 is a mass spectrum showing the distribution of expected cleavage products according to one exemplary embodiment of the invention.

FIG. 5 is a mass spectrum showing the generation of expected cleavage products according to one exemplary embodiment of the invention.

FIG. 6 is a series of mass spectra showing the cleavage pattern generated using exemplary elevated reaction condition temperatures.

FIG. 7 is a mass spectrum showing the distribution of oligonucleotide cleavage products in a non-oxidatively stressed oligonucleotide conjugate sample processed according to a method of the present invention.

FIG. 8 is a mass spectrum showing the distribution of oligonucleotide cleavage products in an oxidatively stressed sample processed according to a method of the present invention, including the presence of degradation products.

FIG. 9 is a diagram of a sample oligonucleotide conjugate, indicating the presence of an unmodified nucleotide cleavage site, wherein “m” indicates a 2′-O-methyl modified nucleotide, “f” indicates a 2′-Fluoro modified nucleotide.

### 5. DETAILED DESCRIPTION

### Definitions

As used herein, and unless otherwise modified, the term “oligonucleotide conjugate” refers to a composition comprising an oligonucleotide and a high molecular weight compound.

The oligonucleotide may be single or double-stranded. In addition, the oligonucleotide may be comprised of “unmodified” or “modified” nucleotide bases. Unmodified nucleotide bases include the purine bases adenine (A) and guanine (G), and the pyrimidine bases thymine (T), cytosine (C) and uracil (U). Modified nucleotide bases include other synthetic and natural nucleotide bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified nucleotide bases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′ modifications, such as, 2′-Fluoro and 2′-O-methyl.

Oligonucleotides that may be analyzed or processed according to the present invention include, but are not limited to siRNAs, antisense oligonucleotides, aptamers and other structural oligonucleotides such as ribozymes. In one exemplary embodiment, the oligonucleotide is an aptamer. Aptamers are short single or double stranded DNA, RNA, and modified RNA oligomers that fold into a particular secondary and tertiary structure.

As used herein, the term “non-immunogenic high molecular weight compound” refers to a compound of approximately 1000 Da or more that typically does not generate an immune response in vivo. Example of non-immunogenic high molecular weight compounds include polyalkylene glycols, such as polyethylene glycol (PEG); polylactide/glycolde polymers (PLGA); polyaminoamines (PAMAM); polysaccharides, such as dextran; and polyoxazolines (POZ).

In one exemplary embodiment, the non-immunogenic high molecular weight compound is a PEG or a derivative thereof. PEGs can range in size from about 5 to about 200 KD, with typical range of about 10 to about 60 KD for pharmaceutical uses. Linear chain PEGs of up to about 30 KD can be produced. For PEGs of greater than about 30 KD, multiple PEGs can be attached together (multi-arm or branched PEGS) to produce PEGS of the desired size. The oligonucleotide construct may comprise either linear or branched PEGs. Oligonucleotide constructs comprising any suitable PEG molecule known in the art may be utilized with the methods of the present invention.

The high molecular weight compound may be covalently attached to a variety of positions on the oligonucleotide, such as to an exocyclic amino group on the base, the 2′-position of the nucleotide, 5-position of a pyrimidine nucleotide, the 8-position of a purine nucleotide, the hydroxyl group of the phosphate, or a hydroxyl group or other group at the 5′ or 3′ terminus of the oligonucleotide. In one exemplary embodiment, the high molecular weight compound is attached to the 5′ terminus, 3′ terminus, or both. The attachment of the high molecular weight compound to the oligonucleotide can be through direct connection or via a linker.

As used in, and unless otherwise modified, the term “linker” refers to both moieties connecting multiple high molecular weight compounds to a core, such as glycine or glycerol, and moieties connecting the core to an oligonucleotide, as well as moieties that link the high molecular weight compound directly to the oligonucleotide. The linker may comprise a thiodiester (—O—C(O)—S—), thionocarbamate (—O—C(O)—(NR)—S—), siloxane (O—Si(R)₂—O—), carbamate (—O—C(O)—NH— and —NH—C(O)—O—), sulfamate (—O—S(O)(O)—N— and —N—S(O)(O)—N—) morpholino sulfamide(O—S(O)(N(morpholino)-), sulfonamide (—O—SO₂—NH), sulfide (—CH₂—S—CH₂—), sulfonate (—O—SO₂—CH₂—), N,N′-dimethylhydrazine (—CH₂—N(CH₃)—N(CH₃)—), thioformacetal (—S—CH₂—O—), formacetal (—O—CH₂—O—), thioketal (S—C(R)—O—), ketal (—O—C(R)₂—O—), amine (—NH—CH₂ . . . CH₂—), hydroxylamine (—CH₂—N(R)—O—), hydroxylamine (—CH═N—O—), or hydrazinyl (—CH₂—N(H)—N(H)—) group.

Cleavage of the high molecular weight compound from the oligonucleotide is achieved by first mixing the oligonucleotide conjugate with a cleavage reagent. In one exemplary embodiment, the high molecular weight compound is conjugated to the oligonucleotide via a linker. In another exemplary embodiment the high molecular weight compound is conjugated to the oligonucleotide via a linker comprising a carbamate group.

The cleavage reagent comprises a nucleophile suitable for initiating hydrolysis of the high molecular weight compound from the oligonucleotide. In one exemplary embodiment the cleavage reagent is a hydroxide. Suitable hydroxides for use as the cleavage reagent may include alkali and alkaline earth metal hydroxides such as, but not limited to, sodium, potassium, magnesium and calcium. In one exemplary embodiment the cleavage reagent comprises sodium hydroxide. In yet another exemplary embodiment, the cleavage reagent comprises an amine nucleophile. In another exemplary embodiment, the cleavage reagent is ammonium hydroxide

The concentration of the cleavage reagent may range from about 0.10 to about 3.0 M. In one exemplary embodiment the concentration ranges from about 0.10 to about 2.0 M. In one exemplary embodiment the concentration ranges from about 0.10 to about 1.0 M. In another exemplary embodiment, the concentration ranges from about 0.5 M to about 0.95 M. In another exemplary embodiment, the concentration ranges from about 0.5 to about 0.85 M. In another exemplary embodiment, the concentration of hydroxide ranges from about 0.5 to about 0.75 M. In yet another exemplary embodiment, the concentration of hydroxide ranges from about 0.5 to about 0.6 M. In another exemplary embodiment, the concentration ranges from about 0.1 to about 0.5 M. In another exemplary embodiment, the concentration ranges from about 0.1 to about 0.4 M. In another exemplary embodiment, the concentration of from about 0.1 to about 0.3 M. In yet another embodiment, the concentration from about 0.1 to about 0.2 M.

The reaction mixture may be formed by dissolving the oligonucleotide conjugate in an appropriate amount of buffer comprising the cleavage reagent (i.e. re-dissolving lyophilized or precipitated oligonucleotide conjugate in a buffer comprising the cleavage reagent), exchanging a composition comprising the oligonucleotide conjugate into a buffer comprising the cleavage reagent using dialysis, exchange columns, or any other standardized buffer exchange method known in the art. Alternatively, a stock cleavage reagent solution may be titrated into a pre-existing composition comprising the oligonucleotide construct, such as a TBE or TAE buffer, to reach the desired cleavage reagent concentration.

The reaction mixture comprising the oligonucleotide conjugate and the cleavage reagent are then incubated, with or without continued mixing, for about 1 to about 72 hours. In another exemplary embodiment the reaction mixture is incubated for about 1 to about 60 hours, for about 1 to about 48 hours, for about 1 to 36 hours, or for about 1 to about 24 hours. In another exemplary embodiment, the reaction mixture is incubated for about 2 to about 24 hours, for about 4 to about 24 hours, for about 6 to about 24 hours, for about 8 to about 24 hours, for about 10 to about 24 hours, for about 12 to about 24 hours, for about 14 to about 24 hours, for about 16 to about 24 hours, for about 18 to about 24 hours, for about 20 to 24 hours, for about 22 to 24 hours. In one exemplary embodiment, the reaction mixture is incubated for about 1 to about 18 hours, for about 1 to about 12 hours, for about 1 to about 8 hours, for about 1 to about 6 hours, from about 1 to about 4 hours, from about 1 to about 2 hours.

The reaction mixture is incubated at about 4° C. to about 60° C., at about 4° C. to about 55° C., at about 4 to about 50° C., at about 4° C. to about 45° C., at about 4° C. to about 40° C., at about 4° C. to about 35° C., at about 4° C. to about 30° C., at about 4° C. to about 25° C., at about 4° C. to about 20° C., at about 4° C. to about 15° C., or at about 4° C. to about 10° C. In another exemplary embodiment, the reaction mixture is incubated at about 20° C. to about 60° C. In another exemplary embodiment the reaction mixture is incubate at about 20° C. to about 50° C. In yet another exemplary embodiment, the reaction mixture is incubate at about 20° C. to about 45° C. In another exemplary embodiment, the reaction mixture is incubated at about 35° C. to about 40° C.

The reaction can be quenched by placing the reaction mixture on ice, exchanging the oligonucleotide into a suitable storage buffer or NMR buffer, or titration of a corresponding weak conjugate acid sufficient to neutralize the cleavage reagent. Prior to downstream analysis the oligonucleotide may be separated from the cleaved high molecular weight compound using liquid chromatography, electrophoresis, or any other standard separation method known in the art. Alternatively, the reaction mixture can by analyzed directly, for example, by LC-MS.

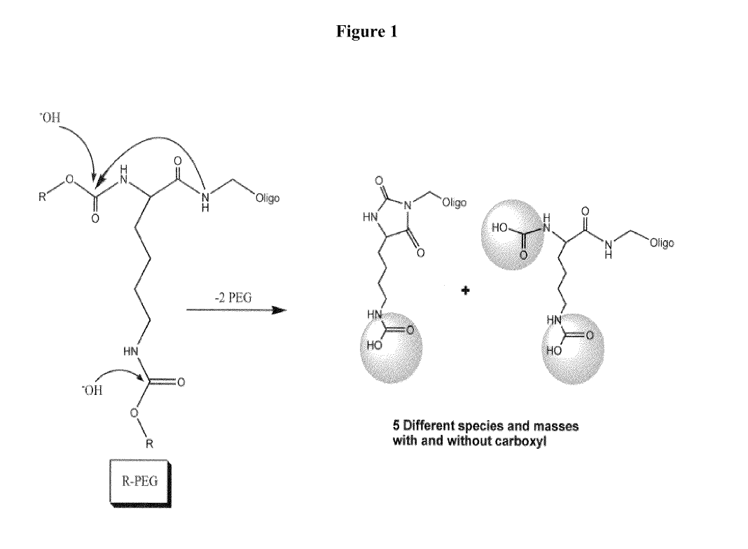

The incubation period results in the formation of distinct cleavage products. In one exemplary embodiment, the cleavage products comprise a high molecular weight compound cleavage product and an oligonucleotide cleavage product. In one exemplary embodiment, the oligonucleotide cleavage product comprises one or more oligonucleotide cleavage product species. The oligonucleotide cleavage product species may comprise detectable residual functional groups at the sites of conjugation. FIG. 1 shows high molecular weight compound cleavage products and oligonucleotide cleavage product species resulting from an exemplary oligonucleotide conjugate processed according to the present invention. The exemplary oligonucleotide conjugate comprises a branched PEG conjugated to an oligonucleotide via a linker comprising a carbamate functionality. The resulting cleavage products include the cleaved PEG molecules and distinct species of oligonucleotides with distinct masses based on the presence of the one or more residual carboxyl groups at the site of conjugation. These residual functional groups give the oligonucleotide cleavage product a distinct mass which can then be detected for example, using mass spectroscopy. Alternatively, the structures may be elucidated by NMR or compared to standards using HPLC.

Oligonucleotide cleavage products resulting from the methods of the present invention can be analyzed by mass spectroscopy in order to detect impurities or characterize degradation products. Mass spectroscopy provides a fast and accurate determination of the absolute molecular weight distribution of a sample. As noted above, oligonucleotide cleavage product species previously conjugated to a high molecular weight compound can contain residual functional groups. When analyzed by mass spectroscopy, the oligonucleotide cleavage product species containing the residual functional groups will register as distinct peaks in the mass spectra from those oligonucleotide cleavage product species not containing the residual functional groups. In this way it is possible to identify oligonucleotide cleavage product species originating from the original oligonucleotide conjugate, other non-conjugate species, and other impurities.

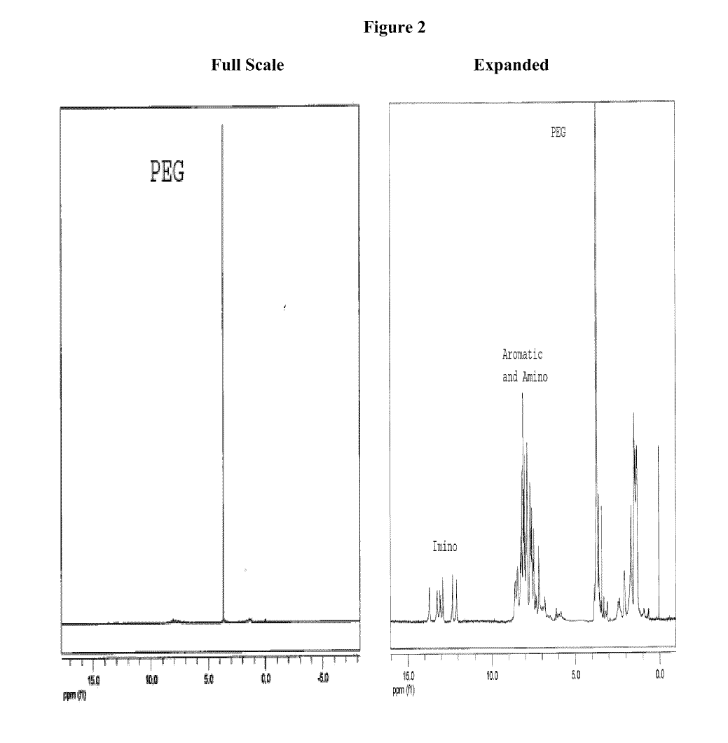

Oligonucleotide cleavage products resulting from the methods of the present invention may also be analyzed by nuclear magnetic resonance in order to detect impurities, or characterize degradation products. The removal of polymer from the oligonucleotide has the added advantage of increasing the sensitivity in NMR. The number of protons from the polymer can dominate the spectra making the detection of low level impurities difficult. In addition, selective excitation can be utilized to suppress the polymer and water signal and detect the oligonucleotide in the presence of a high molecular weight compound (See FIG. 2). While detection of aberrant signals indicative of potential impurities is possible while analyzing the entire oligonucleotide conjugate, it is not possible from analyzing such spectra to readily determine if the aberrant signal is oligonucleotide in nature (i.e. a nucleic acid impurity generated during oligonucleotide synthesis or an oligonucleotide degradation product), or non-oligonucleotide in nature (i.e. high molecular weight compound degradation product or other impurity). Using the method described above, the high molecular weight compound can be effectively cleaved from the oligonucleotide and the oligonucleotide subjected to further analysis to determine the sequence of the oligonucleotide, and/or detect oligonucleotide degradation products through MS or NMR. The NMR spectra in particular can be used in comparison with the spectra of the complete oligonucleotide conjugate to determine if the aberrant peak of the construct spectra correspond to the peaks observed in the spectra of the oligonucleotide alone, wherein detection of corresponding peaks in both spectra indicates the presence of an oligonucleotide based impurity or degradation product and lack of corresponding peaks indicates the presence of a non-oligonucleotide based impurity or degradation product.

Oligonucleotide cleavage products may also be detected by mass spectroscopy. When coupled with mass spectroscopy, the present invention is particularly useful in analyzing degradation products of oligonucleotide constructs containing modified nucleotides. As noted above fragments that where conjugated at their 5′ or 3′ terminus are readily detectable through the presence of residual functional groups at the site of conjugation. Observed aptamer cleavage product species include terminally modified fragments containing a carboxy group at the 5′ terminus, the 3′ terminus, or both, and terminally unmodified cleavage products. The size of the terminally unmodified cleavage products can be determined, for example, the number and location of unmodified nucleotides in the oligonucleotide (See FIG. 9). Detection of additional degradation products, therefore, is indicative of degradation of the oligonucleotide construct. Degradation products may then be further characterized, for example, by sequencing in order to determine where in the oligonucleotide the lesion is occurring.

In most oligonucleotide synthesis protocols it is possible for nucleotide bases to be fully or partially removed from the sugar-phosphate backbone. For example, synthesis of oligonucleotides containing 2′-fluorouridine can result in incorporation of the following impurities into the oligonucleotide:

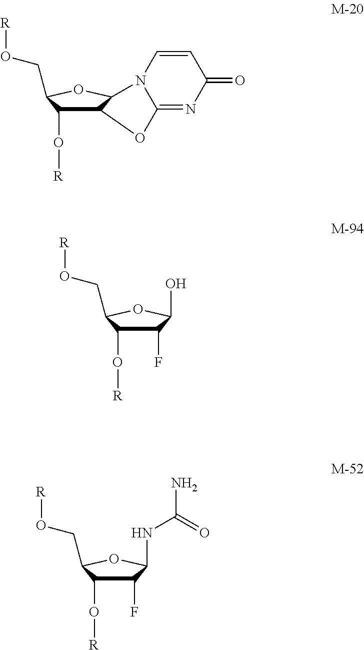

These impurities can still undergo conjugation and therefore can be permanently incorporated into the oligonucleotide conjugate. By providing a means for removing the high molecular weight compound from the oligonucleotide, the present invention provides a means where such impurities can be readily detected using mass spectroscopy and/or NMR.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

### EXAMPLES

### Example 1

A 50 kD PEGylated oligonucleotide was produced via conjugation of a 40 kD branched lysine containing two carbamate linkages at the alpha and epsilon amines of lysine. The conjugation was carried out through the reaction of the activated NHS ester of the lysine and the primary amine of the oligonucleotide. The PEGylated oligonucleotide was subsequently purified by anion exchange and ultrafiltration. The structure of the resulting product is shown below.

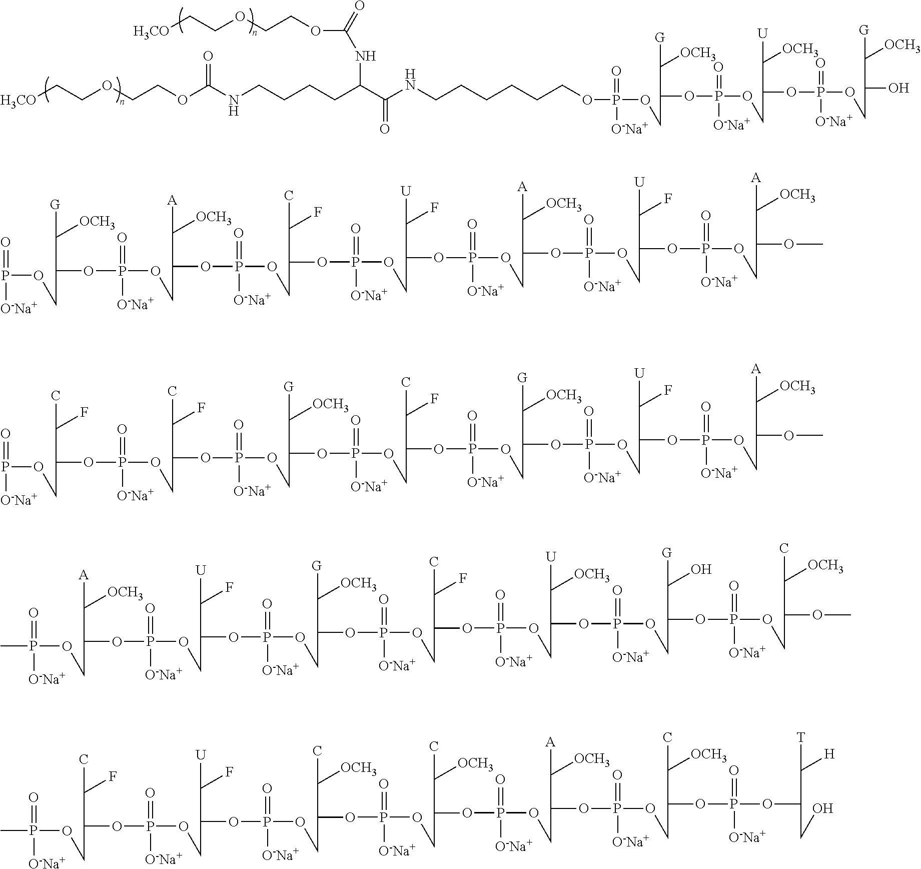

where n is approximately 450

To determine optimal cleavage conditions oligonucleotide conjugate samples (at approximately 21 mg/mL) were exposed to varying cleavage conditions, detailed below in Tables 1 and 2.

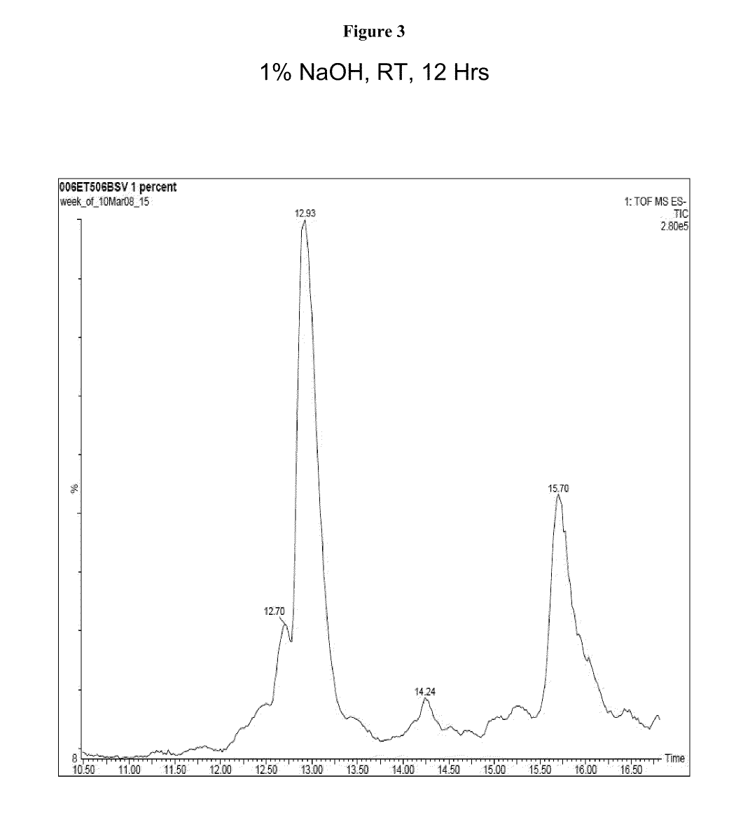

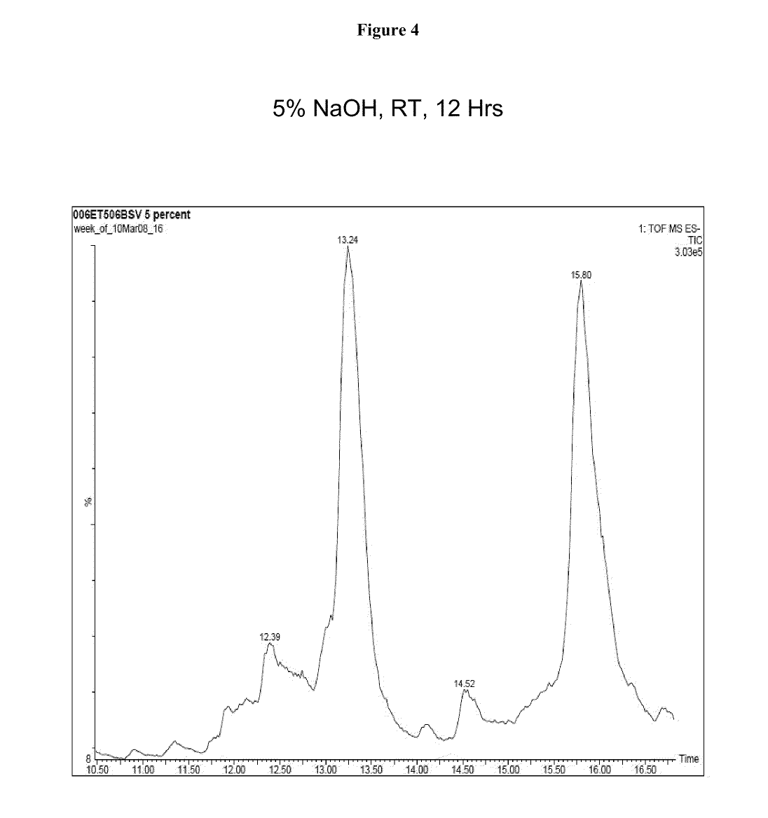

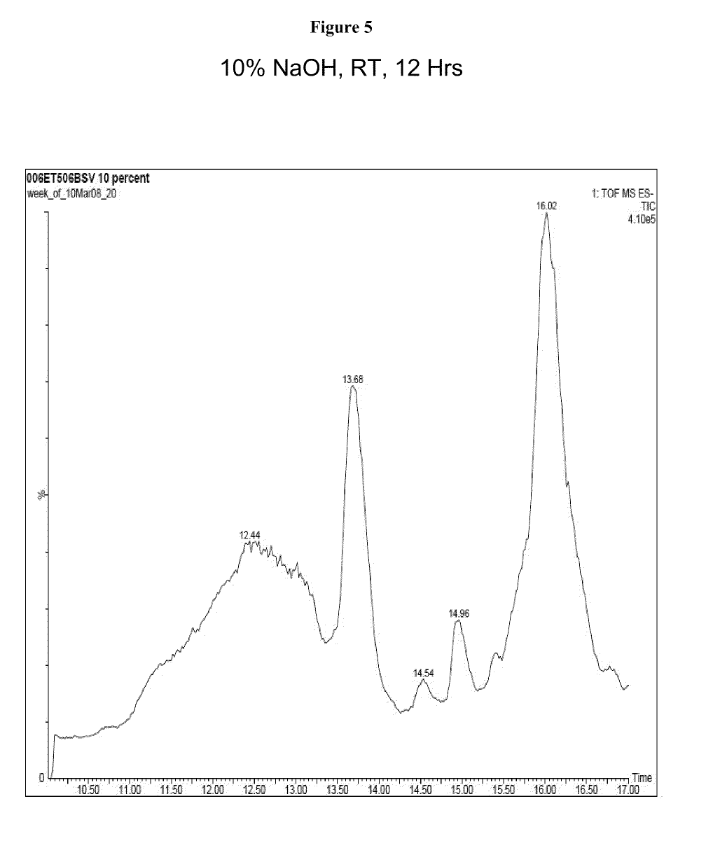

TABLE 1
Incubation at Room Temperature for 12 hours

| Sample No. | NaOH Concentration % | Mass Spec Ref: |
|---|---|---|
| 1 | 1 | FIG. 3 |
| 2 | 5 | FIG. 4 |
| 3 | 10 | FIG. 5 |

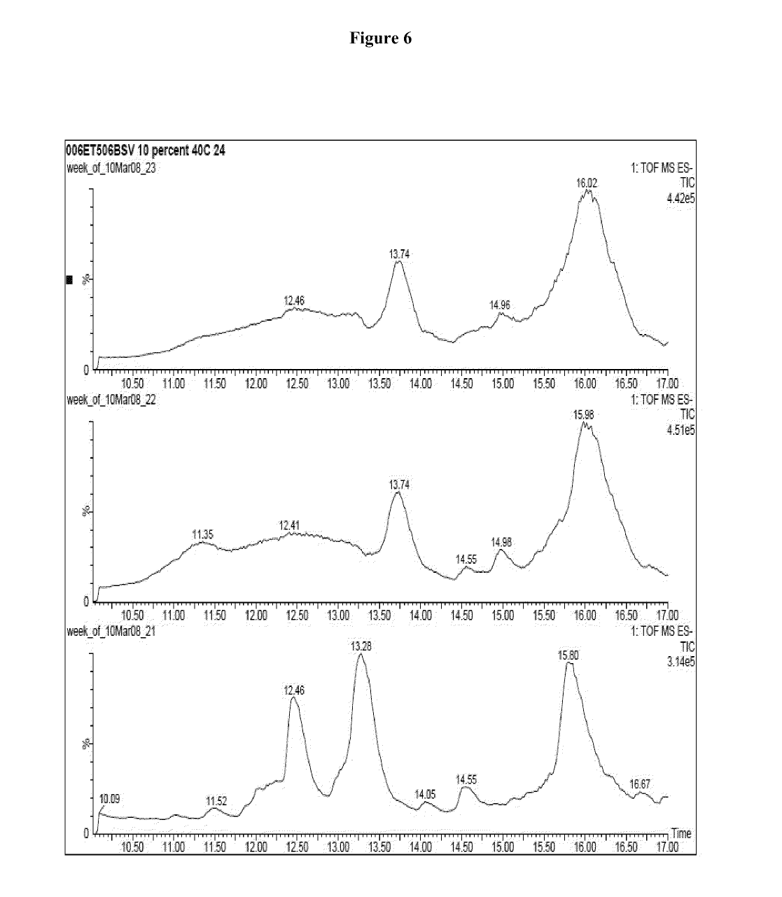

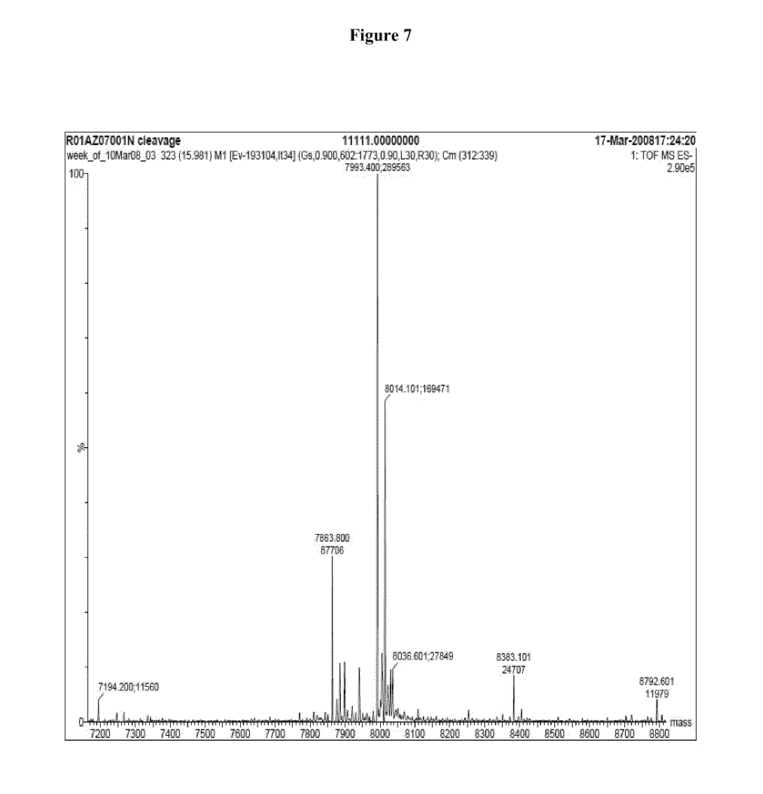

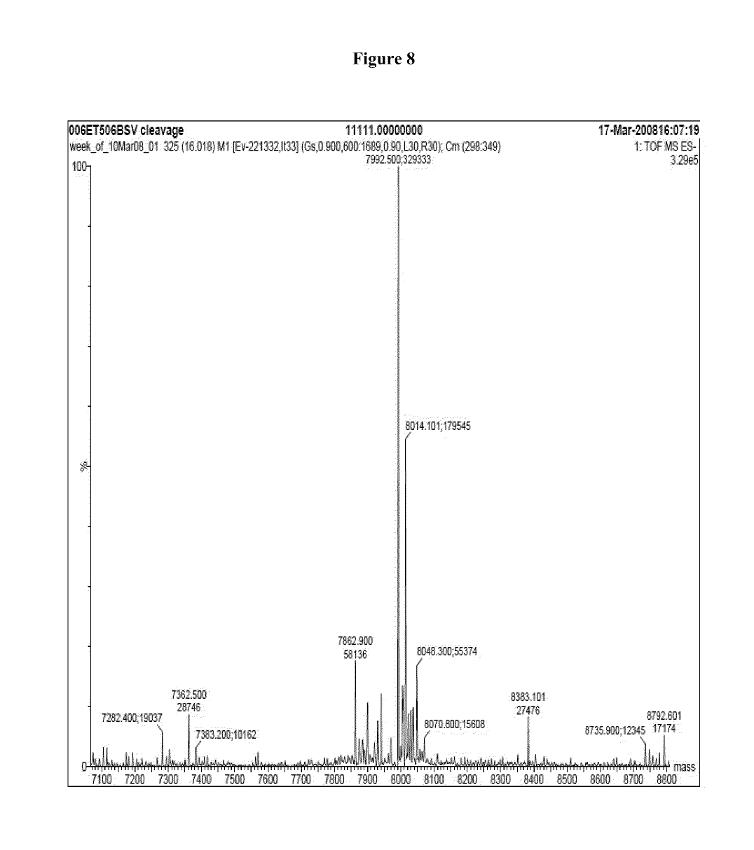

TABLE 2
Incubation at 40° C. for 12 hours

| Sample No. | NaOH Concentration % | Mass Spec Ref: |
|---|---|---|
| 1 | 1 | FIG. 6 |
| 2 | 5 | FIG. 7 |
| 3 | 10 | FIG. 8 |

FIG. 3-5 show the corresponding mass spectra observed under the reaction conditions above. The expected cleavage sites can be predicted based on the structure of the oligonucleotide. The oligonucleotide will cleave at unmodified nucleotide bases. FIG. 9 shows the composition of modified an unmodified bases in the oligonucleotide tested and identifies a riboG cleavage site. As shown in FIGS. 3-5, processing of the oligonucleotide construct according to the method of the present invention resulted in detection of the expected cleavage products without undesirable degradation of the oligonucleotide. Concentrations of 1% and 5% being slightly more optimal than 10%. FIGS. 6-8 show the same cleavage reagent conditions, at an elevated temperature. While, the reaction conditions did result in generation of the expected cleavage products, undesirable cleavage products were also generated. These reaction conditions may not be optimal with the cleavage reagent used, however, it may be possible to modify the reactions conditions based on type of cleavage reagent, concentration of cleavage reagent, and/or length of incubation to increase the selectivity of PEG cleavage at elevated conditions.

To show that the present method can be used to detect degradation products in an oligonucleotide conjugate sample, two samples of the above oligonucleotide conjugate were prepared. On sample was exposed to oxidative conditions, the other maintained as a non-treated control. The two samples were then processing by incubating the samples in 1% NaOH, at room temperature, for 12 hours The samples were then processed by LC-MS to determine the distribution of cleavage products. As shown in FIG. 7, the non-treated control generated the expected cleavage products. The oxidatively stressed sample, however, show a number of elevated cleavage product species as shown in FIG. 8, indicating the presence of degradation products in the sample.

1. A method for removing a high molecular weight compound moiety from an oligonucleotide conjugate comprising:
mixing a composition comprising an oligonucleotide conjugate with a cleavage reagent, to form a reaction mixture; and
incubating the reaction mixture at suitable conditions to generate one or more high molecular weight compound cleavage products and an oligonucleotide cleavage product.

2. The method of claim 1 wherein the cleavage reagent is an alkali or alkaline earth metal hydroxide, or an amine nucleophile.

3. The method of claim 2, wherein the cleavage reagent is sodium hydroxide.

4. The method of claim 1, wherein the cleavage reagent is at a concentration of about 0.15 M to about 2 M.

5. The method of claim 1, wherein the oligonucleotide conjugate comprises a linker connecting the high molecular weight compound to the oligonucleotide.

6. The method of claim 5, wherein the linker comprises a thiodiester (—O—C(O)—S—), thionocarbamate (—O—C(O)—(NR)—S—), siloxane (O—Si(R)₂—O—), carbamate (—O—C(O)—NH— and —NH—C(O)—O—), sulfamate (—O—S(O)(O)—N— and —N—S(O)(O)—N—) morpholino sulfamide(O—S(O)(N(morpholino)-), sulfonamide (—O—SO₂—NH), sulfide (—CH₂—S—CH₂—), sulfonate (—O—SO₂—CH₂—), N,N′-dimethylhydrazine (—CH₂—N(CH₃)—N(CH₃)—), thioformacetal (—S—CH₂—O—), formacetal (—O—CH₂—O—), thioketal (S—C(R)—O—), ketal (—O—C(R)₂—O—), amine (—NH—CH₂—CH₂—), hydroxylamine (—CH₂—N(R)—O—), hydroxylamine (—CH═N—O—), or a hydrazinyl (—CH₂—N(H)—N(H)—) group.

7. The method of claim 6, wherein the linker comprises a carbamate intermediate to a PEG and a core.

8. The method of claim 6, wherein, the linker comprises a carbamate group intermediate to the oligonucleotide and PEG.

9. The method of claim 1, wherein the oligonucleotide cleavage product comprises one or more residual functional groups at one or more sites of conjugation.

10. The method of claim 8, wherein the linker comprises a carbamate group and the oligonucleotide cleavage product comprises one or more carboxy groups at the sites of conjugation.

11. The method of claim 1, wherein the high molecular weight compound comprises a non-immunogenic high molecular weight compound.

12. The method of claim 1, wherein non-immunogenic high molecular weight compound comprises a linear or branched polyethylene glycol (PEG).

13. The method of claim 1, wherein the reaction mixture is incubated for about 24 hours at about 4° C. to about 60° C.

14. The method of claim 1, wherein the reaction mixture is incubated for about 24 hours at about 40° C.

15. The method of claim 4, wherein the cleavage reagent is sodium hydroxide and is present in the reaction mixture at a concentration of about 0.16 M to about 1 M.

16. The method of claim 1, further comprising separating the high molecular weight compound cleavage product from the oligonucleotide cleavage product.

17. A method of analyzing a sample comprising an oligonucleotide conjugate comprising:
mixing the sample with a cleavage reagent to form a reaction mixture;
incubating the reaction mixture at suitable conditions to generate one or more high molecular weight compound cleavage product and an oligonucleotide cleavage product;
separating the high molecular weight compound reaction product from the oligonucleotide cleavage product; and
analyzing the oligonucleotide cleavage product to determine one or more physical characteristics selected from the group consisting of; the oligonucleotide cleavage product's sequence, and the presence of degradation products, and the presence of impurities in the oligonucleotide cleavage product.

18. The method of claim 17, wherein the cleavage reagent is an alkali or alkaline earth metal hydroxide.

19. The method of claim 17, wherein the cleavage reagent is sodium hydroxide.

20. The method of claim 17, wherein the cleavage reagent is at a concentration of about 0.15 M to about 1 M.

21. The method of claim 17, wherein the oligonucleotide conjugate contains a linker connecting the high molecular weight compound to the oligonucleotide.

22. The method of claim 21, wherein the linker comprises a thiodiester (—O—C(O)—S—), thionocarbamate (—O—C(O)—(NR)—S—), siloxane (O—Si(R)₂—O—), carbamate (—O—C(O)—NH— and —NH—C(O)—O—), sulfamate (—O—S(O)(O)—N— and —N—S(O)(O)—N—) morpholino sulfamide(O—S(O)(N(morpholino)-), sulfonamide (—O—SO₂—NH), sulfide (—CH₂—S—CH₂—), sulfonate (—O—SO₂—CH₂—), N,N′-dimethylhydrazine (—CH₂—N(CH₃)—N(CH₃)—), thioformacetal (—S—CH₂—O—), formacetal (—O—CH₂—O—) thioketal (S—C(R)—O—), ketal (—O—C(R)₂—O—), amine (—NH—CH₂—CH₂—), hydroxylamine (—CH₂—N(R)—O—), hydroxylamine (—CH═N—O—), or a hydrazinyl (—CH₂—N(H)—N(H)—) group.

23. The method of claim 21, wherein the linker comprises a carbamate group intermediate to a core and the PEG.

24. The method of claim 21, wherein the linker comprises a carbamate group intermediate to the oligonucleotide and the PEG.

25. The method of claim 17, wherein the oligonucleotide cleavage product comprises one or more residual functional groups at one or more sites of conjugation.

26. The method of claim 24, wherein the linker comprises a carbamate group and the oligonucleotide cleavage product comprises one or more carboxy groups at the sites of conjugation.

27. The method of claim 17, wherein the high molecular weight compound moiety comprises a non-immunogenic high molecular weight compound.

28. The method of claim 27, wherein non-immunogenic high molecular weight compound moiety comprises a linear or branched polyethylene glycol (PEG).

29. The method of claim 17, wherein the reaction mixture is incubated for about 24 hours at a temperature ranging from about 4° C. to about 60° C.

30. The method of claim 17, wherein the reaction mixture is incubated for about 24 hours at a temperature of about 40° C.

31. The method of claim 19, wherein the cleavage reagent is sodium hydroxide and is present in the reaction mixture at a concentration of about 0.16 M to about 1 M

32. A method of confirming the structural integrity of an oligonucleotide conjugate comprising:
a) mixing the oligonucleotide conjugate with a cleavage reagent to form a reaction mixture;
b) incubating the reaction mixture at suitable conditions to generate one or more high molecular weight cleavage products and an oligonucleotide cleavage product;
c) separating the oligonucleotide cleavage product from the high molecular weight product;
d) analyzing the oligonucleotide cleavage product for the presence of one or more degradation products in the oligonucleotide cleavage product, wherein the presence of one or more degradation products indicates degradation of the oligonucleotide.
